(19) United States
(12) Patent Application Publication
(10) Pub. No.: US 2012/0209373 A1
(43) Pub. Date: Aug. 16, 2012

(54) Coated Stint

(76) Inventors: Martina Schmitt, Garstedt (DE); Manfred Gulcher, Raesfeld-Erie (DE)

(21) Appl. No.: 13/265,043

(22) PCT Filed: Apr. 16, 2010
(86) PCT No.: PCT/EP10/02353

§ 371 Date: Mar. 14, 2012

(30) Foreign Application Priority Data
Apr. 18, 2009 (DE) 10 2009 018 013.3

(51) Int. Cl. A61F 2/06

(52) U.S. Cl. 623/1.43

(57) ABSTRACT

The invention relates to a stent, in particular intended for cardiovascular applications, provided with a bioresorbable polymer coating containing a restenosis-inhibiting agent, with said stent below its polymer coating having a metal surface which is smoothed by electropolishing and passivated and into which carbonium ions are implanted, with said polymer coating containing a (homo- or copolymer of hydroxycarboxylic acids which is terminally esterified.

The invention relates to a stent provided with a bioresorbable polymer coating laden with an active agent capable of inhibiting restenosis, with the stent comprising a metallic material exhibiting a passivated surface smoothed by electropolishing into which carbon ions have been implanted. In particular, the stent is meant for cardiovascular use.

More often than not, stents are employed when vascular bottlenecks have to be eliminated. This is most frequently necessary to treat a stenosis of coronary arteries caused by arteriosclerosis. Arteriosclerosis may be due to various causes, for example may develop as a result of a vascular injury giving rise to a hyper-proliferation of the vascular epithelial cells. In consequence, this may lead to is more or less severe local narrowing of the blood vessel involved, and even an obliteration of the vessel may occur causing the necrosis of tissue areas to which the relevant vessel supplies blood. In the coronary area such an occlusion leads to cardiac infarction.

Vascoconstrictions in the area of the coronary arteries can be treated in various ways. One option in this context is to perform bypass surgery, which is to be considered a more or less severe operative intervention. Vascoconstrictions may also be remedied endovascularly by balloon dilatation, for which purpose the constricted location of the vessel is dilated by means of a hydraulically inflatable balloon. Such a vessel dilatation as a rule involves the placement of a stent, a process during which the stent (“vessel prop”) is crimped onto the balloon and then dilated together with the balloon and placed in the constricted location of the vessel. The stent serves to support the tissue and, ideally, keeps the vessel open. These treatment options are also known as “percutaneous transluminal coronary angioplasty” (PTCA) and “stenting” and are performed in an endovascular manner which is much easier on the patient than a surgical intervention and treatment of a vascoconstriction.

Stents placed in position with the help of inflatable balloons are pressed against the vessel wall at significant pressure which is normally considerably higher than 6 bar. The pressure load thus exerted on the vessel wall may cause injuries to the wall triggering a complicated series of processes, details of which have not been completely cleared up to this day. Among other things, in the framework of the so-called neointimal hyperplasia proliferation and migration of the medial and intimal vascular smooth muscle cells occur which ultimately results in tissue to accumulate and become attached to the stent. This leads to restenosis, i.e. the reoccurrence of a vessel constriction will be encountered.

Attempts have been made in various ways to counteract such a restenotic vessel narrowing, however, these proved to be partially successful only. To reduce the damage caused to the vessel wall, one method attempts to keep the pressure load low when placing the stent. However, since a certain minimum pressure has to be exerted to widen the vessel this approach has its limits. There are, nevertheless, certain areas where success could be achieved with self-expanding stents, for example those made of nitinol material.

Another widely adopted approach involves the careful smoothing of the stent surface by means of polishing processes to rule out injuries due to burrs and unevenness. For this purpose stents are mechanically or electro-chemically smoothed. In this case as well there are certain limits, however, which cannot be exceeded because crimping a stent onto a balloon and subsequently expanding the stent during placement lead to surface irregularities.

Another factor to be considered is that especially in the coronary area the vessels are embedded into muscle tissue. Due to the fact that muscle tissue is “in motion” the vessel wall is subjected to constant strains in the form of mechanical friction at the locations in contact with the stent. The stimulation mechanism thus effected contributes to restenosis.

Of special significance in the context of restenosis is the stent surface, in particular the inner surface of the stent, because it may be conducive to the accumulation of cell tissue and/or sclerotic substances. Due to this reason the stent surface must not only be designed to be smooth and repellent to prevent undesirable material from accumulating but it must as well be passivated in such a manner that the surface will not cause additional stimulation to the injured vessel. For this purpose, aside from treating the stent surface by electropolishing, foreign ions are introduced, for example, to achieve an enrichment and thus passivation of the surface. In particular, the implantation of carbon ions can render the metal surface to be more biocompatible and reduce the discharge of toxic ions, for example nickel ions stemming from nickel-containing medical steel material.

Just another approach to counteract the restenosis hazard is to make use of proliferation-inhibiting medical substances, for example Paclitaxel or Rapamycin. These medical agents, for example, may be applied to the vessel wall by means of balloons coated with these medicaments. Another possibility involves the coating of stents with such proliferation-inhibiting agents. In the pursuit of this, suggestions were made to provide the stent with suitable pits or channels accommodating the agent. More frequently employed are, however, medicament-containing coatings consisting of biocompatible polymers (resomers), including those that are resorbed by the body in the course of time. Such coatings cause the medical substance to be released over a time period of varying duration.

It has been learned from practical experience that the restenosis risk is highest in the days and weeks shortly after date vessel dilatation was carried out. It is thus of primary importance to make available a proliferation-inhibiting medical substance first and foremost during this time span. It is to be noted, however, that the suitability of many carrier materials is only limited when it comes to reliably release the medical substance uniformly over a defined period of time.

A problem existing with many bioresorbable polymer coatings applied to stents used as medicament substrate is that the coatings do not reliably detach from the stent surface. Bioresorbable polymers of a type for example put on the market under the trade name of Resomer® by the company of Boehringer Ingelheim are polar substances that due to interaction effects attach to the metal surface more or less firmly, especially if the surface has not been meticulously polished and passivated. The fact that steel used for medical purposes tends to cause oxygen to oxidatively accumulate on its surface also leads to polar polymers to increasingly and more firmly attach to the surface. It thus follows that the detachment of the polymer and accordingly its resorption by the body can only be poorly controlled.

Taking all these aspects into account, it is thus the objective of the present invention to provide a stent that not only has low-level stimulus properties but, moreover, is capable of releasing a proliferation-inhibiting medical substance of a resorbable polymer in appropriate doses over a limited time period, and enables a low restenosis rate to be achieved. The stent shall be highly biocompatible on a long-term basis, and its design shall enable the attending physician to place it without extra effort and expense in a customary manner and by usual methods.

As per the invention this objective is reached by providing a stent of the kind first mentioned above, with the polymer coating which accommodates the medicament load containing a homopolymer or copolymer which stems from hydroxy-carboxylic acids and is terminally esterified.

The inventive stent consists of a customarily employed metallic material, for example steel suitable for medical uses or nitinol. The stent itself is given a surface smoothed by electropolishing, such surface being additionally passivated. The passivation treatment may be carried out in that the stent during electropolishing is suspended in a medium suited for this purpose and serves as anode.

Electropolishing expertly performed may significantly improve in particular the roughness characteristics of a smoothed surface. As a result, the real surface characteristics come close to those considered ideal. Adopting mechanical smoothing techniques enables real-to-ideal surface characteristics ratios to be achieved that range between 100 and 200 and even with the most efficient methods this can hardly be improved. Well performed electropolishing will lower the ratio of real vs. ideal surfaces to values below 10. If the ratio of a real compared to an ideal surface ranges between 2.5 and 5 it is considered ideal which means the surface available for accumulations on the stent surface (after the polymer coating has been removed) will be 2.5 to 5 times larger than that of the ideal surface. Accumulations are thus reduced and lower restenosis rates achieved. Moreover, a surface smoothed in this manner will have a lower stimulus potential affecting adjacent body tissue.

In addition to its surface being smoothed by electropolishing the near-surface areas of the stent are modified by ion implantation. For ion implantation purposes carbon ions have proved most suited. Ion implantation is effected in a manner known per se. A description of a stent treated by carbon ion implantation with carbon ions of suitable distribution can be found in DE 202 20 589 U1.

Carbon ion implantation involves the introduction of carbon ions into the near-surface layers of the stent, but does not mean the stents are coated with carbon material. The implantation of ions causes saturation of free valences of the metal constituents of the stent and results in oxygen to be displaced. Maximum carbon contents are not detected at the surface itself but some μm below. At the surface itself, the proportion of oxygen atoms and in particular also of alloying additions such as nickel is significantly reduced. In accordance with DE 202 20 589 A1 this effect is turned to practical account in that the release of toxic ions from the stent into the blood is lowered and the biocompatibility of the stent surface enhanced.

However, in the light of the present invention this effect just plays a secondary role. The primary objective focuses on deactivating the near-surface area of the stent as well as reducing the near-surface oxygen content considerably. Near-surface oxides of stent metals lead to the incorporation of water and attachment of polar substances, in particular also from a polymer coating, so that, consequentially, via hydrogen bonding and, as the case may be, ionic bonds such a polymer coating firmly adheres to the surface, especially if the coating consists of polar materials. In the event of bioresorbable polymer layers which require resorption and detachment from the stent surface to be reliably achieved such a relatively firm connection with the stent is undesirable for the purpose in mind. The polymer adheres to the surface of the stent for too long a time with the consequence that the time period during which the medical agent is released is too long as well. This is counteracted by reducing the bonding energy arising between the stent surface and the polymer.

The implantation of carbon ions usually takes place after the stent surface has been processed by electropolishing. The electropolishing process causes surface unevenness to be leveled out and in this manner results in the overall surface to diminish. If electropolishing is performed under oxidative conditions—as a rule the object to be polished is connected as anode—this will also cause an oxide-rich surface to be generated. Such an oxide-rich surface is not only very biocompatible but also conducive to reducing the release of toxic ions. The extraordinarily smooth surface furthermore minimizes the accumulation of biological materials, in particular cell material. On the other hand and as has been described earlier, there is a better attachment of polar polymer coatings.

Bioresorbable polymer coatings are known for stent technology purposes. In many cases polyester is used. According to the invention inner polyesters are employed which are based on hydroxycarboxylic acids. These may be present both in the form of homopolymers and copolymers, with the copolymers being statistical or block copolymers. In any case, the polyesters are additionally terminally esterified.

In principle, polyesters on the basis of hydroxycarboxylic acids are known and can be produced, for example, through polymerization or coopolymerization of hydroxycarboxylic acids, their dimeric esters or by means of lactones. Suitable are, in particular, hydroxycarboxylic acids with up to 8 carbon atoms including glycolic acid, lactic acid, hydroxybutyric acid and hydroxyvalerianic acid. As a result of their structure the polymers and copolymers of hydroxycarboxylic acids are excellently suited as medicine administering carriers, they are very biocompatible and excellently bioresorbable. The polyesters on the basis of glycolic acid and lactic acid are preferably produced through the polymerization of glycolide and lactide.

The polyester coatings put to use in accordance with the present invention do no longer contain free carboxyl groups. In fact, terminal carboxylic acid groups and as the case may be also pendant carboxylic acid groups are, in essence, all esterified. Used as esters in this case are in particular esters of alcohols with up to 6 carbon atoms, especially ethyl esters.

Due to the esterification the free carboxyl groups of the polymer coating these groups are prevented from forming bonds with the stent surface, either by the formation of salt or the formation of hydrogen bonding. At the same time, the stimulus potential of the polycondensates modified in this way is significantly reduced. Especially in the early phase of implanting a stent it appears to be expedient to avoid any kind of additional stimulation.

Special preference is given to copolymers of lactic acid and glycolic acid, for example at a ratio of between 25 and 75 mole % of lactic acid and 75 to 25 mole % of glycolic acid. Preferred is a ratio ranging between 45 and 55 mole % of lactic acid and 55 and 45 mole % of glycolic acid, but in particular a copolymer of about the same mole fractions. The lactic acid may be present in its D-form or L-form, however, preferably as a roughly equimolar mixture of D- and L-lactic acid, in particular according to the formula:

Where,

x denotes the number of lactide units, y the number of glycolide units and n the number of repeat units in the polymer.

By means of esterification processes known per se the terminally esterified hydroxycarboxylic acid polymers can be produced from unesterified basic materials which are commercially available. Preferred is an ethyl esterified product which through esterification is obtained from Resomer® RG 504 and is commercially available (company of Boehringer Ingelheim).

The polymer coating of the inventive stent contains a restenosis-inhibiting, in particular proliferation-inhibiting, antiangiogenic, anti-inflammatory or anti-thrombotic active agent of a nature as has many times been described in literature. Preferred active agents are Rapamycin and Paclitaxel. The active agent is integrated into the polymer coating in a manner known per se and released by migration processes as well as degradation of the polymer coating.

The polymer coating containing the medicine can be applied to the prepared stents in a customary way, for example by means of dip coating or spray coating methods. In both cases the stent is provided with the desired coating of the designated thickness by repeated wetting with and drying of a solution containing the polymer and active agent.

The surface treatment of the stents according to the invention should be performed in such a manner that after carbon ion implantation at least 50% of the bonding activity in relation to the polymer coating is saturated through the implanted carbon ions. Preferably, more than 75% of the bonding activity should be saturated. The implantation of carbon ions into the near-surface layers will not only result in the content of toxic (nickel) ions in the boundary layer to decrease but as well cause the metal oxides and embedded oxygen to be neutralized such that the accumulation of water is diminished and the interaction between the polar groups of the polymer coating and the metal surface is greatly reduced. As regards the distribution of carbon ions in the stent surface, express reference is made here to FIG. 2 of DE 202 29 589 U1.

On the whole, it has been found that by combining a number of steps in the treatment of the stent surface—involving in particular careful electropolishing and including the oxidative passivation as well as implantation of carbon ions—with a special resomer coating, the carboxylic acid functions of which having been neutralized by esterification, a stent can be provided which in terms of the release of its medicine load and degradation of its polymer coating meets the respective requirements. After the polymer coating has been removed, a very biocompatible and restenosis-inhibiting stent surface is left and has properties conducive to the ingrowth into the vessel wall.

1. Stent, in particular intended for cardiovascular applications, being provided with a bioresorbable polymer coating containing a restenosis-inhibiting agent, with said stent below its polymer coating consisting of a metal surface which is smoothed by electropolishing and passivated and into which carbonium ions are implanted, characterized in that, the polymer coating contains a homo- or copolymer of hydroxycarboxylic acids which is terminally esterified.

2. Stent according to claim 1, characterized in that the polymer coating consists of a homo- or copolymer of lactic acid and/or glycolic acid.

3. Stent according to claim 1, characterized in that the polymer coating comprises 25 to 75 mole % of lactide units and 75 to 25 mole % of glycolide units.

4. Stent according to claim 3, characterized in that it comprises 45 to 55 mole % of lactide units and 55 to 45 mole % of glycolide units.

5. Stent according to claim 4, characterized in that the polymer coating consists of approximately 50 mole % of D,L-lactide units and approximately 50 mole % of glycolide units.

6. Stent according to claim 1, characterized in that the polymer of the polymer coating is terminally esterified with a C1-C6 alcohol.

7. Stent according to claim 6, characterized in that the ester is an ethyl ester.

8. Stent according to claim 1, characterized in that at least 50% of the bonding activity of the stent's metal surface is saturated by carbon ions.

9. Stent according to claim 8, characterized in that at least 75% of the bonding activity of the stent's metal surface is saturated by carbon ions.

10. Stent according to claim 1, characterized in that an active agent has been incorporated into the bioresorbable polymer coating.

11. Stent according to claim 10, characterized in that the active agent is a proliferation-inhibiting agent.

12. Stent according to claim 11, characterized in that the active agent is Rapamycin or Paclitaxel.

13. Stent according to claim 1, characterized in that it consists of medical steel or nitinol.

14. Stent according to claim 1, characterized by a ratio of <10 of the real in relation to the ideal surface after electropolishing.

15. Stent according to claim 14, with a ratio between the real and ideal surface ranging between 2.5 and 5.
